(19) United States
(12) Patent Application Publication
(10) Pub. No.: US 2012/0042546 A1
(43) Pub. Date: Feb. 23, 2012

(54) Orthotic Device and Method of Manufacture

(76) Inventor: Stephanie Cosgrove, Queensland (AU)

(21) Appl. No.: 13/257,356

(22) PCT Filed: Dec. 18, 2009
(86) PCT No.: PCT/IB09/55844

§ 371 Date: Nov. 11, 2011

(30) Foreign Application Priority Data
May 20, 2009 (AU) 2009902160

(51) Int. Cl. A61F 5/14

(52) U.S. Cl. 36/140

(57) ABSTRACT

An orthotic device for use with an individual's footwear is customised to the individual's therapeutic, comfort and footwear requirements by using, as a template for its manufacture, an existing orthotic device that has been previously created for that individual. The therapeutically contoured shape of the existing device is captured in a malleable layer that sets to be self-supporting, so to allow creation of a new therapeutic layer that replicates the surface of the existing device and that forms a therapeutic surface of the new device.

### FIELD OF INVENTION

This invention relates to a method of fabricating an orthotic device for location into or onto an item of footwear to provide a therapeutic mechanical benefit to the wearer. The invention also relates to a method of reproducing the profile of an existing orthotic device for incorporation into a new, replacement or supplementary orthotic device. The invention extends to an orthotic device having a composite structure and a reproduced functional profile.

### BACKGROUND TO THE INVENTION

Custom-made orthotic devices may be prescribed for an individual by a podiatrist, physiotherapist, chiropractor or other medical, allied health or associated practitioner or technician for use within a shoe. Such devices are often removable and made of plastic, rubber or other material, designed to be movable between, and usable in, different pairs of shoes. Custom orthotics are made for an individual to address a physical problem pertaining specifically to their foot and have an upper surface that is shaped to deliver a desired therapeutic outcome to such individual. In shaping the surface, some reference is made to the foot, perhaps by way of a mould or any other modelling tool, be it physical or electronic. Other pre-fabricated shoe inserts are also known to be called orthotics, although it is not universally accepted that this use of the term is proper where the devices are not customised to the wearer. These off-the-shelf shoe inserts are commonly made in a selection of sizes, densities or other variants, but without distinction with respect to an individual person. The present invention is directed to custom orthotic devices.

Regular orthotic devices are known to be made using either a computer-aided design and computer aided manufacturing (CAD-CAM) system or a thermoplastic sheet system. Those comprising an orthotic plate, made using CAD-CAM, do not need to be comprised of thermoplastics, although they usually are. In using a CAD-CAM system, the design is created like a topographical map and sent to a router that carves a block of plastic out to the desired shape without the need for heating and forming of a sheet of uniform thickness Thus, CAD-CAM orthotics are not uniform in thickness. Thermoplastic sheets for the more traditional making of orthotics will be heated to approximately 170° C., under which conditions they are malleable and formable. They are then applied to a replica of the foot (often made in plaster of Paris) that has been modified to create the desired therapeutic effect. This plaster replica is then put sole side up in a vacuum-forming device (former) with the malleable, heated sheet above it. The former's bladder is pulled around the replica by the vacuum function and thus the malleable sheet adopts the same contours as the modified replica. The thermoplastic is allowed to cool while still vacuum-pressed against the replica and, once formed, can then be removed for machining. This device, although not yet complete, would be able to support the weight of the designated user without deforming more than is planned for. It is common for some deformation to be ‘built into’ a plate by selecting the material a little thinner than it would need to be to remain unyieldingly solid when used by the envisaged person for the anticipated level of activity. By making it ‘not solid’, the degree of flex allowed gives added shock absorption. At this point of the process, the plastic is of uniform thickness (for example, 4 mm), which is most problematic when considering areas under the heel and back from the 5th metatarsal head. A sheet this thick has to be thinned in these areas without exception, as that degree of thickness would create a ‘high heel’ effect at the rear and an uncomfortable and probably unwearable drop-off for the foot at the front (or toe end) of the device.

The thickness of a custom-made orthotic of the prior art is typically in the range from 3.5 to 5 mm and this relatively large thickness is a factor in the poor fitting ability of such orthotics into sleek shoes.

Often there is a disparity between the dimensions of the resultant orthotic made according to measurements of the foot or one selected off the shelf and the internal dimensions of the shoe that is the preferred footwear of the wearer. For example, an ordinary orthotic will typically not fit into a slim-line feminine shoe or a low-cut football boot.

It is, therefore, an object of this invention to provide a method of manufacturing a customised orthotic that will fit a much broader range of footwear than orthotics manufactured by prior methods and will provide more desirable options for consumers in their choice of orthotic-capable footwear.

It is also an object of the invention to provide a method of custom-manufacturing an orthotic device for an individual to their individual specifications.

It is further an object of the invention to provide a method of manufacturing an orthotic device by reproducing the profile of an existing such device, while modifying other factors related to volume and density of the resultant new device.

It is another object of the invention to provide an orthotic device manufactured without the need to directly measure or examine the user's foot.

Another object of the invention is to provide a method of manufacture of an orthotic device that has minimal thickness and therefore will not serve to elevate the wearer's foot within the shoe, such elevation being a consequence that would commonly result in slippage of the shoe at the heel.

It is also an object of the invention to provide an orthotic device having reduced bulk in comparison with prior orthotics, thus fitting into an expanded range of footwear.

The preceding discussion of the background to the invention is intended to facilitate an understanding of the present invention. However, it should be appreciated that the discussion is not an acknowledgement or admission that any of the material referred to was part of the common general knowledge in Australia or elsewhere as at the priority date of the present application.

Throughout the specification and claims, unless the context requires otherwise, the word “comprise” or variations such as “comprises” or “comprising”, will be understood to imply the inclusion of a stated integer or group of integers but not the exclusion of any other integer or group of integers.

### SUMMARY OF INVENTION

The method of manufacturing an orthotic according to the invention does not rely on the use or availability of the foot of the intended user, but rather on the use of an existing orthotic that the user has already commissioned from another source, or of a model or other representation, such as a cast, of the orthotic to be reproduced.

Thus, according to existing first aspect of the invention, there is provided a method of manufacturing an orthotic device for use with a desired item of footwear, the method comprising the steps of providing an existing orthotic device that has an existing, therapeutically-shaped contoured surface, and using said existing surface as a template in forming a body that has a contoured surface that replicates said existing surface substantially entirely.

In a preferred form of the invention the method includes forming a cast of said existing surface.

Preferably, the cast is formed by applying a shape-capturing material in sheet form to said existing surface and allowing it to set to a self-supporting state, so that a surface of the sheet that is not in contact with the existing surface, defines a dorsal surface that replicates said existing surface.

In an alternative form of the invention, the method includes applying a thin sheet of malleable shape-capturing material to the cast and allowing said sheet to set to a self-supporting state, so that a surface of the sheet that is in contact with the cast, defines a plantar surface that replicates said existing surface.

In a preferred embodiment, the method includes separating the sheet, once set, from the surface to which it has been applied.

In a further preferred form of the invention, the sheet comprises a thermoplastics material.

The invention preferably further includes the step of adapting the set sheet to define an orthotic device that has been customised for use with the desired item of footwear. The method includes customising the sheet to fit the foot-receiving space of the footwear.

In an embodiment, the step of adapting the sheet includes identifying an area of the said existing orthotic device that is not therapeutically essential to the functioning of the device and identifying a corresponding area of the sheet, so as to define a peripheral zone thereof. The peripheral zone thus comprises a part of the sheet that does not have an imperative effect on the functional proficiency of the device.

In an embodiment, the method further includes diminishing the peripheral zone to a minimum practical thickness. In further embodiment, the method includes reducing the surface area of the peripheral zone.

In a preferred embodiment, the corresponding area is selected from an area adjacent a lateral edge of the device, a lateral margin of an anterior edge of the device, an area below the central heel and a combination of any of the aforesaid.

In a preferred form of the invention, the method comprises providing the peripheral zone with a profile that tapers from being relatively thick where it merges with the therapeutically-shaped surface, to being relatively thin at an opposed exterior edge. The method thus includes forming the peripheral zone as an area of relatively reduced thickness and removing bulk from it to produce a profile that tapers away progressively from a relatively thicker functional area of the sheet, which comprises the replicated therapeutic shape of the existing device, and ends in a fine edge.

In an embodiment, the peripheral further zone comprises a comfort/cosmetic layer applied to the sheet. The thickness of the sheet may optionally be reduced, particularly when intended to be permanently adhered to a surface in the foot-receiving space of an item of footwear.

Preferably, the supporting means is selected to resist flexion of the sheet under the force exerted by the weight of the user in use. In a preferred embodiment, the supporting means comprises a reinforcing layer.

In a further preferred embodiment of the invention the method includes filling with a filler, at least partially, a void otherwise defined between the plantar surface of the device and a surface of the desired footwear.

In a particularly preferred form of the invention, the filler comprises a closed cell polymer. Reinforcing and filler layers will be absent from the peripheral zone as they are not functionally required. Further, the method includes affixing supporting means to the plantar surface of the sheet.

In a further form of the invention, the method includes applying a shape-capturing material to the first therapeutically-shaped and contoured surface, causing the material to assume an impression of the contoured surface thereon and allowing the material to form a body that has a surface permanently shaped according to the contoured surface and separating the body from said existing surface.

In a further preferred form of the invention, the shape-capturing material is settable and the method includes allowing the material to set to form a body that has a surface bearing an impression of the contoured existing surface permanently thereon.

In a still further preferred form of the invention, the method comprises adapting the body to define an orthotic device customised to the dimensions of a target item of footwear with which it is intended to be worn by a user.

In a further preferred form of the invention, the method comprises adapting the body to provide a workpiece for shaping into an orthotic device and shaping said workpiece to be customised to the dimensions of a target item of footwear with which the device is intended to be worn by a user.

In a preferred embodiment, the method includes providing the settable material in the form of a malleable sheet. The sheet has a lower or plantar surface on which the contours are replicated and an upper or dorsal surface opposed to the lower surface. Further, the contours are preferably replicated on both the upper and the lower surfaces.

In a particularly preferred form of the invention, the sheet material is a thermoplastics material. The thermoplastics material thus provides a workpiece having shape memory, which may be used to capture the therapeutic contours onto itself, usually by means of softening via heating, then by forming the material to the required contour shape, then cooling it to cause it to retain the new shape on the surface of the workpiece. The workpiece then, being of substantially uniform thickness, serves as a membrane over the dorsal (upper) surface of the existing orthotic device. When it is removed from contact with the original device, it retains the shape of that device.

In an embodiment, the method further comprises the step of forming a peripheral zone, this being an area of minimal thickness and minimal contour in zones identified as not being imperative to the function of the finished device.

Preferably, the method further comprises providing the peripheral zone with a profile that tapers from the therapeutically contoured surface toward an internal surface of the footwear on which the device is to rest in use.

In an embodiment, the workpiece has a plantar (lower) surface on which the contours are captured and an upper surface opposed to the plantar surface.

In a further embodiment, the upper and plantar surfaces both capture the contours. In this embodiment, the workpiece is typically a sheet of thin material in which the shape of the dorsal surface corresponds to that of the plantar surface.

Preferably, the method of the invention includes rendering the workpiece malleable and forming the workpiece, while malleable, to the existing orthotic, thereby to assume the contours on the dorsal surface and allowing the workpiece to set, thereby losing its malleability so as to define substantially permanently a therapeutically contoured layer.

In a further preferred form of the invention, the method includes determining the suitability of the existing orthotic to receive the malleable workpiece material intimately enough to capture its contours directly. If the determination is that the workpiece material is unsuitable, the method optionally includes the step of making a mould using a different but suitable material that is capable of capturing the contours of the existing orthotic device without harming said device.

Where volume reduction is the primary reason for commissioning of a new orthotic device, the method includes reducing the bulk of the workpiece after the contours have been captured.

In an embodiment, the method includes applying filler material to the plantar surface of the workpiece to fill, at least partially, voids anticipated between such surface and an internal surface of the footwear on which it is intended to rest while in use.

Optionally, the method includes the step of forming contours on the plantar surface of the filled device, such contours being determined by considering the contours of the internal surface of the footwear.

In a further preferred embodiment, the method includes the step of applying a comfort-providing layer to the dorsal surface of the workpiece, such layer being of a substance selected to not affect the therapeutic contours of said surface. Cosmetic affect can also be considered. It is not necessary that such comfort-providing layer be possessed of shape memory.

Preferably the comfort-providing layer is thin in comparison with the workpiece. In an embodiment, the comfort-providing layer is less than 2 mm thick. Preferably, the comfort-providing layer is about 1 mm thick.

In a preferred embodiment, the comfort-providing layer is a material that is resilient to use with feet and footwear,

Preferably, the method includes adapting the workpiece to facilitate a smooth transition between the therapeutically contoured surface and the internal aspects of the footwear. In a further embodiment, this may include extending the comfort layer to adhere to aspects of the target footwear to improve cosmesis of the device/footwear unit when complete.

In a preferred form of the invention, the method includes laminating a supportive substrate to the underside of the workpiece. Preferably, the substrate is selected to provide a desired degree of mechanical strength for the composite material comprising the substrate and the workpiece. In an embodiment, the substrate is selected to provide a desired degree of flexibility for the composite material. The method includes bonding the substrate to the workpiece.

According to a second aspect of the invention, there is provided a method of manufacturing a new orthotic device, the method comprising the steps of providing an item of footwear with which an intended user desires to use the new device, determining design parameters for the new device to enable use thereof with the desired footwear, providing an existing orthotic device used by said user and having a therapeutically-shaped dorsal surface, providing a sheet of malleable material, replicating the therapeutically-shaped surface of the existing orthotic device permanently on a surface of said sheet and adapting the shaped sheet to fit comfortably with the desired item of footwear according to the user's preference, to define the new device.

The design parameters may include the shape, configuration, structure, materials and dimensions.

In an embodiment, the step of determining the parameters comprises collecting data relating to one or more of the size, shape and configuration of the desired footwear item and weight of the user.

In a preferred form of the invention the step of adapting the sheet includes one or more of trimming its size, adding a supporting portion, reducing its bulk and adding filler material between the sheet and a surface of the desired footwear item.

According to a third aspect of the invention, there is provided an orthotic device comprising a sheet of shape-retaining material having a dorsal, therapeutically-shaped surface that replicates a therapeutically shaped surface of an existing orthotic device used by said individual.

Preferably, the device has been adapted to fit the foot-receiving space of a predetermined item of footwear of the individual.

In a preferred form of the invention, the device is customized by adapting it to derive structural strength from the structure of the footwear with which it is to be used.

The device is adapted to complement said internal contours preferably by determining the internal contours of said footwear and extracting data that characterises said internal contours.

In an embodiment, the sheet is substantially uniform in thickness. The thickness is preferably in the range from about 0.8 mm to 2.5 mm and further preferably in the range 0.9 mm to 1.6 mm.

In a preferred form of the invention, the sheet has a peripheral zone corresponding to a therapeutically non-essential area of the existing orthotic device.

In an embodiment, the peripheral zone is sacrificed in the interests of space saving. Typically the zone is located at a lateral margin and mid-heel of the device and is rendered as thin as desired by the user, having given consideration to the desired footwear.

Preferably, the peripheral zone has a profile tapering from being relatively thick where it merges with the therapeutically shaped surface to being relatively thin at an opposed exterior edge

In a preferred embodiment, the peripheral zone is created when the thermoplastic sheet has a profile tapering from being full thickness in the areas that are essential to the therapeutic function of the device to a thinnest practical endpoint on its outermost margin. The thinned areas are those considered functionally expendable. Preferably, the profile tapers toward an internal surface of the footwear on which the device rests in use.

Preferably, the thickness of peripheral zone is less than about 2 mm. Further preferably, the peripheral zone tapers to a thickness of less than 1.0 mm at its outer edge. Most preferably, the peripheral zone tapers to a thickness in the range from 0.5 mm to 1.2 mm at its outer edge.

In a further embodiment, the device is of elongated form and has width not exceeding of the width of the desired item of footwear with which it is to be used.

Further preferably, the sheeting is chosen to have properties selected from hardness, softness, flexibility and rigidity and combination thereof, and which have been determined according to user requirements.

In a further embodiment, the device may be optionally adhered to the footwear. This may be done to inhibit its movement in the foot-receiving space in use or to provide additional strength to a finer device.

In a further preferred embodiment, the device further has supporting means located in abutment to the sheet. Preferably the supporting means comprises a reinforcing layer for the sheet.

According to a fourth aspect of the invention, there is provided an orthotic device for insertion into a user's footwear, the device comprising a body having a therapeutically contoured dorsal surface and a lower portion adapted to fit the foot-receiving space of a particular shoe of the user.

In a preferred embodiment, the dorsal surface replicates the therapeutic shape of an existing orthotic device of the user as a result of direct contact having been established during shaping of said surface with a surface selected from a therapeutically shaped dorsal surface of said first orthotic device or a surface derived from said first device surface.

Preferably, the device is customized by adapting it to match closely the internal dimensions of the footwear into which it has been designed to be inserted. When congruently matched, and optionally adhered, the strength of the footwear materials to which it is aligned will add to the strength of the device. In a preferred embodiment, the device comprises adhesion means for securing it against movement in the footwear when fitted. When incongruently matched to leave a void in the arch section of the plantar aspect of the device, flexibility will be increased allowing this aspect of manufacture to be used to further customise the rigidity of the device

Further, the device optionally includes a comfort-providing and cosmetic layer applied to the therapeutically contoured dorsal surface.

The comfort-providing layer comprises a material that is resilient under pressure from the foot of the user in use.

According to a further preferred embodiment of the invention, the orthotic device body is reinforced by at least one structurally fortifying layer.

In an embodiment, the reinforcing layer is located below the plantar surface of the body in use.

In a fifth aspect of the invention a method of manufacturing an orthotic device, comprises the steps of providing an existing orthotic device having a desired therapeutic profile comprising a contoured surface, making a cast of the surface, providing a thermoplastics sheet in malleable and settable state, applying the sheet to the surface of the casted replica so as to cover the surface area representative of the original device so as to take on the contours thereof, allowing the sheet to set to a state in which it retains the desired contours in the shape of its surface, removing the sheet from the casted replica, applying a filler to the sheet and optionally adapting the sheet to customise it for fitting into a particular desired item of footwear of an intended user.

According to a sixth aspect of the invention, a method of manufacturing an orthotic device to replicate the shape of an existing, therapeutically-contoured surface of an existing orthotic device, comprises the steps of providing a sheet of malleable shape-retaining material, introducing it into intimate contact with a surface selected from the said existing surface of the existing device and a surface derived therefrom, so as to cause a surface of the sheet to assume the shape of the existing surface, retaining the assumed shape in the sheet surface so that the sheet is self-supporting when separated from the selected surface and separating said sheet therefrom.

In a preferred form of this aspect of the invention, the derived surface comprises a cast of the existing surface.

### BRIEF DESCRIPTION OF DRAWINGS

In order that the invention may be readily understood, and put into practical effect, the invention will now be described with reference to the accompanying figures in which:

FIG. 1 presents in (a) a dorsal plan view of a left orthotic device, the therapeutically contoured surface of which is to be replicated by the method of the present invention, in (b) a posterior-to-anterior view of a cross section taken along the line A-A′ in (a) and, in figures (c), (d) and (e), a similar cross sectional view of an orthotic device manufactured according to a preferred embodiment of the method of the invention.

FIG. 2 is a schematic diagram of an alternative process for producing an orthotic device according to the invention.

FIG. 3 illustrates from the medial aspect, a longitudinal-section view of an embodiment of an orthotic device made according to the invention.

FIG. 4 is a schematic plantar plan view of an embodiment of an orthotic device made according to the invention with the inclusion of a filler material.

### DETAILED DESCRIPTION OF EMBODIMENTS

The initial step in the manufacture of an orthotic device that replicates the therapeutically functional contours of an existing orthotic device according to the invention is to obtain or provide an existing orthotic that has a surface shape defined by the desired therapeutically functional contours. The existing orthotic then is used as a template in the manufacture of the new device in which the therapeutic surface is replicated in the contours of the new device. This method therefore is not designed to reproduce a replica of the existing orthotic device as a whole: It is concerned with reproducing the shape of the therapeutic functional dorsal surface only. This replicated surface can then be combined with a set of structural features designed to suit the comfort and footwear requirements of the user. These features may be introduced to improve on the comfort to the user over that of the existing device. They may alternatively, or in addition, be introduced to customise the new device to be worn with a particular item of the user's footwear, such as a low-cut football boot, feminine shoe, running shoe, or an open sandal. The customised features include modifications made to the depth, breadth, flexibility and density of the device when compared to the existing device. The said modifications are thus primary reasons why a new device is sought, enhancement of the comfort of the user when using the footwear of the user's choice being a key objective.

An assessment of the condition of the functional surface of the existing orthotic is desirably carried out to ascertain which of the embodiments of the method available under this invention to employ in replicating its surface for the new device to be manufactured. These contemplate direct capture of the existing therapeutic surface by the material from which the new device is to be formed, or utilizing an additional step to capture the contours to a mould from which the new device will be fashioned. If the existing device has a dorsal surface that is in good condition, a direct capture method may be used. If the covering is buckled, torn or otherwise compromised, it may need to be stripped off. The contour-capturing step of the invention can then be completed on the ‘naked’ orthotic and a new cover reapplied to the functional orthotic surface later, so as to recondition it for further use, if required. If the material of the functional surface of the existing orthotic is very delicate but in good condition, it may need to be protected, removed and subsequently replaced, or have a mould made of the existing orthotic as an interim step in the method of contour capture used, as discussed below.

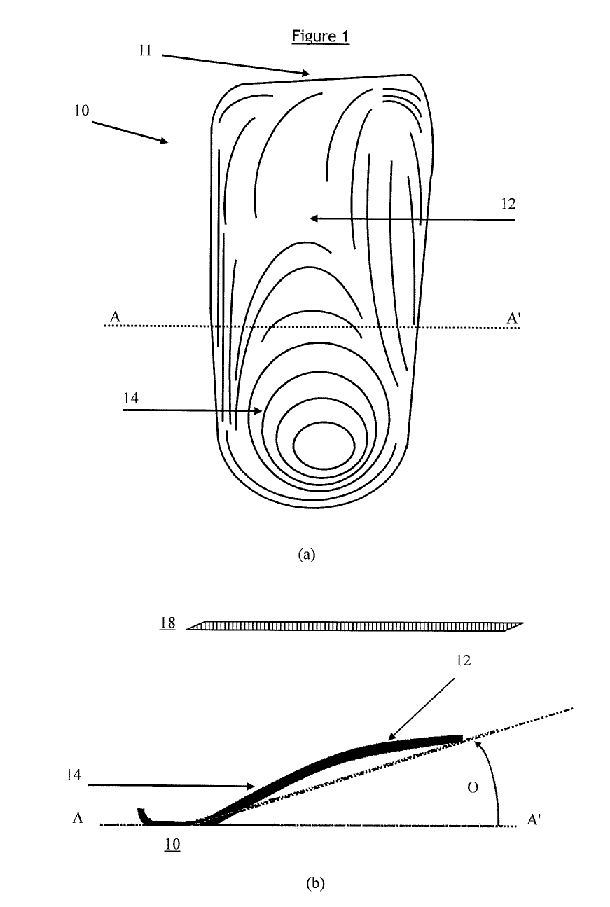

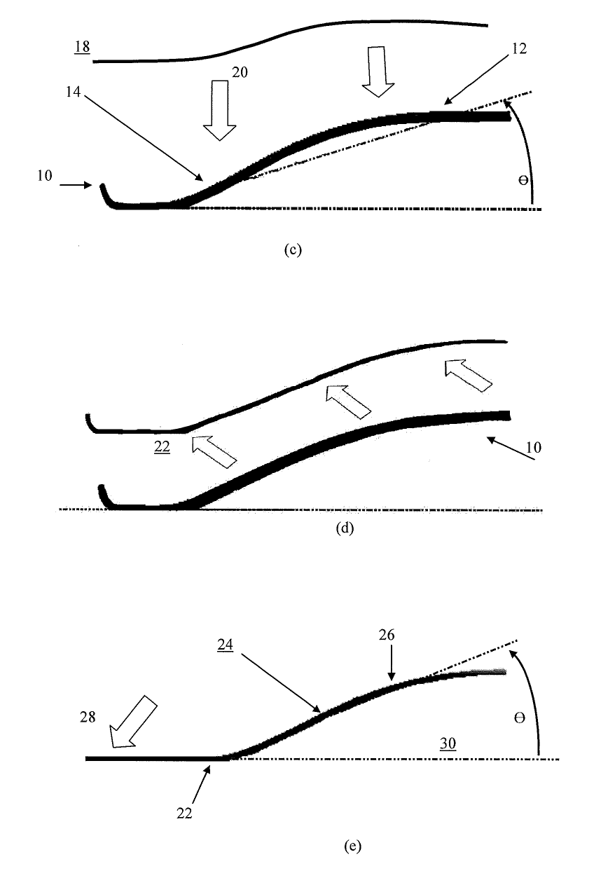

The assessment optionally may involve further surveying of the existing orthotic, in particular to determine the angle that the upper contour of the orthotic makes relative to the ground (determined by use of a convenient flat surface such as a table top) and note it for reference in producing the new device. This noted angle of the original orthotic will be referred to as ⊖° and is set by the original prescriber. With reference to the angle ⊖ in FIG. 1(b), this is often in the range 4° valgus (everted) to 8° varus (inverted), with 4° varus being a favoured therapeutic prescription

With the assessment complete, the next step of the process is to extract contour data from the existing device. This may be done by replicating it onto a different surface, which may either be a sheet of material possessing shape memory that will become part of the end product, or an interim mould, as will be described in the alternative direct contact processes below.

Thus, in the first alternative for performing the replication, a sheet of material possessing shape memory is applied to the therapeutic surface of the existing orthotic, so as to cause it to take on the latter's shape and retain the contours captured therein. Preferably, the sheet comprises a settable material that, after capturing the shape of the dorsal surface of the existing device, is allowed to set so as to retain the shape permanently, subject to the allowance of a predetermined degree of resilience when in use. A suitable example of a settable material is a thermoplastics substance. However, the settable material may alternatively be one that sets on heating (thermosetting), or one that is caused to harden to a set state through the application of an external or additional stimulus, in particular a chemical hardener or external electromagnetic radiation, such as ultraviolet rays. The replication is found to be optimal when the sheet is pressed firmly against the existing orthotic surface so as to be in close contact with it over its entire therapeutic area. Typically, the therapeutic area coincides with substantially the entire dorsal surface.

In cases when the settable material is thermoplastic sheeting, the sheet is heated to be malleable, then pressed against the therapeutically functional surface of the existing orthotic so that it closely follows the surface contours, and then is allowed to set by cooling, thereby to capture in its shape the contours against which it has been in abutment.

The thermoplastic sheet material is selected to have a relatively low temperature at which it becomes malleable. Preferably, it should not require heating to above 100° C. as this may have a deleterious effect on some types of orthotic covers found in existing devices (the purpose of this invention not being to destroy the existing orthotic if it still has therapeutic value for the user). Preferably, the temperature is about 80° C. The sheet should not be malleable at less than about 50° C., so as to avoid accidental deformation, such as may occur if, for example, it is left in a hot parked car. However, it will be appreciated that any other appropriate sheet material capable of contour memory would be suitable for use (subject to safety or toxicity considerations).

The sheet that captures the therapeutic contours and is included as a component of the finished device should be made as thin as is practical, taking into consideration the strength requirements for the intended individual user. The minimum requirement is for this new sheet (‘the body’ of the new orthotic), once set, to provide strength enough to hold the contours it captures from the existing orthotic. An assessment of its strength and flexibility or rigidity with regard to the weight of the end user may be carried out at this point. Further materials may optionally be laminated on to it if additional strength is required, the strength being a function of the choice of materials. Alternatively, it may be required to stand alone and be self-supporting.

The thermoplastic sheet providing the therapeutically functional portion of the device is preferably substantially uniform in thickness, the thickness being preferably in the range from about 0.8 mm to 2.5 mm and more preferably from about 1.0 mm to 1.6 mm and most preferably from about 1.2 mm to 1.4 mm. However, it is to be appreciated that selected areas of the sheet may, if desired, be reduced in thickness, for example by grinding it slightly thinner after forming. The areas selected will be often from the central heel and of the lateral aspect of the device relating approximately to the area under the 5th metatarsal. This thickness reduction step may be performed to adapt the formed sheet to fit a particular item of footwear, or in accordance with other user requirements. Preferably, the reduction of bulk does not exceed 0.5 mm of thermoplastic material removed. However, when the sheet is appropriately thin and already meets the space saving requirements of the intended footwear, certain cases may require no thickness reduction to be performed.

It is important to know the approximate weight of the wearer to determine the densities of materials to employ in the new device. Depending on anticipated weight-bearing requirements, the body of the new orthotic device may be reinforced by at least one structurally fortifying layer. Preferably, the reinforcing layer is located below the plantar surface of the sheet comprising the body in use. Weight must also be evaluated relative to several other factors: For example, the longer the arch span of the foot, the stronger the material would need to be to withstand the same body mass pressures. Also, the expected or intended level of activity of the user when using the device should be considered.

The dimensions and durometer of the existing orthotic are desirably assessed in concert with the interior dimensions and construction properties of the target footwear, with a view to providing the customer with an idea of the likely potency of the finished device. For example, a customer may decide to proceed with an orthotic of the invention in sandal “A” that, it is estimated, can be made 90% as potent in terms of its therapeutic effect as the original device. This person may decline to proceed with modification suited to sandal “B” if it is known that, due to different shoe properties, it can only be made to a relative potency of 50%. It will be appreciated that the traditional orthotic device of the prior art will either fit in the user's shoe or will not, with significant modifications generally not possible. Whether it fits will be by way of coincidence rather than by design.

By contrast, consider that the method of the invention enables production of an orthotic made to any point on the continuum from 0% as potent as the original device (for example a flat piece of thermoplastic), to 100% of the desired level of forcefulness possessed by the original device. If the device at 100% efficiency then fits the shoe the user wants to wear, no further adaptation is needed. It should be noted that producing such a ‘100%’ device does not necessitate constructing a direct copy of the original orthotic as it may still be possible through the use of variant materials to change the nature and dimensions of the device without an impairment of function. If it is considered that the device made to that ‘100%’ specification will not fit the desired footwear, consideration is given to what dimensions and qualities the device could possess while satisfying the intended user. To allow fuller appreciation of this concept, consider a woman with constant foot pain who works in a professional or business environment where she seeks to wear heels every day. This person may decide that it is to her advantage to commission the making of a 60% effective orthotic that will suit her dressing style. Another such example is a woman who is getting married may order for a single use a customised orthotic to be made according to the invention because she wants to be able to dance at the reception wearing her chosen wedding shoes where the alternatives of limiting her activity or compromising her footwear choices are not palatable to her It is an aspect of the invention that the experienced manufacturer will become adept at estimating the relative efficiency of the device given the co-consideration of the user's age, weight, foot length, efficiency of the existing orthotic, shoe preferences, intended use, desired lifespan and availability of the option to adhere the device into the footwear.

As an alternative to the direct method of capturing the functional contour of the existing orthotic described above, the method involves forming an interim mould of the therapeutic surface of the existing orthotic device to be replicated and then manufacturing a new device by shaping the material that will provide the new therapeutic surface by using the mould as template. The material of the mould needs to retain shape memory under mild conditions not deleterious to the surface integrity of the existing orthotic. Non-limiting examples of suitable shape-retaining materials for forming the mould include plaster of Paris, latex and deformable foam blocks. A thin layer may be used to protect the original device from soilage by plaster, for example, plastic food wrap.

The material for the new device (often thermoplastic) is then used to capture the required orthotic contour from the interim mould, thus forming the therapeutic surface piece. This method is advantageous in cases where it is found that the covering material of the template orthotic is too delicate to tolerate direct copying by application of a heated layer, such as a layer of the thermoplastic.

It can thus be seem that the two alternative approaches described in the paragraphs above differ in that, in the first, direct approach, the material capturing the contour becomes a workpiece that is incorporated into the resulting product device as a component part. In the second approach, the material applied to the existing orthotic is used as a mould only: a contour-capturing material is then applied to this mould to capture the shape for the therapeutic surface of the final product. The material of the mould does not itself become a component part of the final product. At the end of this step and irrespective of the approach used, a workpiece comprising a material imbued with the same therapeutic shape, defined by the contours of the dorsal (top) surface of the existing orthotic device, has been generated.

Once the desired surface contours have been captured in the shape of the workpiece, the next step in forming the new orthotic device is to adapt the workpiece by providing it with structural features to suit specific requirements of the end user, taking into consideration factors that may include the comfort desires of the user, the size and weight of the user, the need for fortification of the workpiece for use in a particular service, such as sporting activity, and the spatial or size parameters of a particular footwear item of the user.

Preferably, the device is customized by adapting it to match the foot-receiving space, including the internal dimensions of the footwear with which it has been designed to be used or worn. If congruently matched, the strength of the footwear materials to which it is aligned will add to the strength of the device. Where footwear is possessed of a contoured foot-supporting surface, such as an arch support, the workpiece being fashioned into the device may be shaped to reflect this contour exactly and thus be perfectly congruent and without voids, resulting in a stiffer orthotic plate. If more material is removed, more flex will occur in the plate. If no material were removed from the plantar aspect of the workpiece to adjust for the presence of an arch support, the device would teeter and be unstable in the footwear.

Adhesion of the device to the footwear may also have a significant effect on strength, rigidity and lifespan. By way of illustration, consider an arched piece of sheet metal possessed of some flexibility. Where the arch is placed, concave side down, on a table top and a loading force applied, the material will be significantly more flexible where the ends are untethered and allowed to move away from each other. If the ends were fixed to the table, the sheet would be much more resistant to such a downward application of force. Thus, the manufacturer of the device of this invention has many factors to consider in striving to design the perfect device.

Depending on the thickness and strength of the workpiece, particular structural features may optionally be added: By way of non-limiting example, further material is affixed to its plantar (bottom) aspect to provide an appropriate balance between strength and flexibility, suitable for its intended use, user and footwear. The material may comprise a further layer of a substance having a desired functional property, for example, rigidity, flexibility, resilience, impact absorption or damping, odour elimination and interfacial grip.

When the functional material is added to the workpiece, a laminated structure is formed. Further layers having desired properties may be added to satisfy the end user's service and comfort requirements. Non-limiting examples of materials to use for reinforcement of the workpiece include a further plastic layer, for example ethylene vinyl acetate or similar, a further thermoplastic substance, a foam, for example injected expanding foam, rubber, leather, cork, silicon, latex, metals, various engineered substances, fibrous board or card.

It is advisable to affix such further layers to the plantar (rather than dorsal) surface of the therapeutic portion so as not to diminish the integrity of the copied therapeutic surface contours of the workpiece.

Beneath the arch of the device and between the underside (plantar surface) of the reinforcing layer and the surface of the footwear with which it is to be used and may exist a scalloped-out cavity referred to as a void. The void is optionally filled, at least partially, with suitable filler material. The filler material is preferably shock absorbing and has rebound properties. The filler is selected to prevent the finer upper material of the reinforcing layer from collapsing under repeated application of body weight with each step.

Optionally the filler material may have adhesive properties that serve to anchor it against movement within and in relation to the surfaces of the footwear. Non-limiting examples of suitable materials for the filler include gels, gums, latex, silicon, rubber, foam rubber or combinations of the aforementioned. In an embodiment, the filler material has adhesive properties that secure it permanently in place in the footwear. In a further embodiment, a double-sided adhesive tape may be applied to the plantar surface of a filler that is not itself adhesive, so that the orthotic is caused to adhere to the dorsal aspect of the sole of the footwear.

Optionally, a finishing layer is applied to the dorsal (top) surface of the functional portion for the purposes of cosmesis and as a comfort barrier between the skin of the end user and the therapeutically contoured surface. The presence of this cosmetic finishing layer is advantageous when the material of the therapeutic surface has the potential to be less comfortable against the skin of sensitive patients than materials not possessed of such contour memory. Materials providing a greater degree of comfort in the cosmetic layer include textiles, leathers, rubbers, foams (such as a polyurethane foam) or any other natural or synthetic material suited to the application. The comfort-providing layer is therefore desirably chosen according to considerations of compression resistance, friction coefficient, lifespan, weight, thickness, odour control and launderability. An example of a suitable material is split thickness pig leather.

Optionally, this layer should also be selected or adapted to create a seamless transition between device and footwear by continuing beyond the surface of the orthotic and adhering onto the internal surface of the footwear item.

To adapt the workpiece to suit the comfort requirements of the user, the method of the invention optionally includes identifying an area of the original orthotic device being replicated that is not therapeutically essential and diminishing a corresponding area in the sheet, referred to as the peripheral zone. Preferably, the method includes diminishing the peripheral zone to a minimum practical thickness, taking into account at least some of the comfort-enhancing factors mentioned above. This includes removing a least a portion of the peripheral zone or even the entire zone.

Any areas thus determined to be expendable will be reduced to the finest possible thickness or removed entirely, as they do not have an imperative effect on the functional proficiency of the device. These areas, particularly along the lateral border of the device distal to the calcaneus (heel bone), may be spot heated and pushed flat to the plane of the supporting surface. In later machining, these areas may be ground as thinly as desired to achieve optimal fit into the desired footwear. According to the method of the invention, the peripheral zone is diminished in substance to provide an area of very reduced thickness that tapers away progressively from the relatively thicker functional areas that define the therapeutic areas of the thermoplastic material sheet to a finest endpoint.

The peripheral zone is therefore located laterally to the therapeutically contoured portion of the workpiece and has a profile tapering toward the lateral internal surface of the footwear on which the device rests in use. Preferably the thickness of peripheral zone is less than 2 mm and the edge is crafted to fit snugly into the foot-receiving space defined within the target footwear. It is envisaged that in the majority of situations, the therapeutic effect of the device will be supplied primarily by the medial approximately 70% of the dorsal surface of the device. Thus the lateral periphery of the device may be tapered to provide ease of accommodation in footwear.

An example of the process of manufacturing an orthotic device according to the invention is illustrated schematically in FIG. 1. In FIG. 1(a), a device 10, which has been previously fabricated for use with the user's left foot, is shown in plan view. It has an anterior end 11 and a therapeutic dorsal surface 12 that is desired to be replicated in a new device. The dorsal surface possesses contours 14 that were crafted relative to a representation of an individual's foot (be that the physical foot itself, an electronic image or other) to achieve a therapeutic effect on some aspect of the mechanical function of the foot, be it in its stationary or kinetic use. Whilst substantially the whole of the dorsal surface 12 is contoured, gradients vary and certain areas of effect are more vital to the therapeutic success of the device than others. It is the contoured dorsal surface 12, or at least a desired major portion thereof, that is to be replicated in a new device. In the inspection phase of the invention process, it had been previously noted that the existing device made an angle of intersection with the supporting surface of β°. In the cross sectional frontal plane view positioned at FIG. 1a's line A-A′ as shown in FIG. 1(b), the angle is being examined and noted for later replication to orientate the new device in the frontal plane.

The contours 14 are to be captured in the new device by providing a sheet of thermoplastic material 18 and heating it to a malleable, but coherent state and, with reference to FIG. 1(c), applying it in this state, to cover and assume intimate contact with the surface of the therapeutic surface 12 as generally indicated by arrows 20.

With reference to FIG. 1(d), the shaped sheet 20, also shown here in cross sectional frontal plane view, is allowed to cool and harden in situ to form workpiece 22, which is then separated from surface 12 of the existing device 10 as indicated by the direction of the arrows.

With reference to FIG. 1(e), the workpiece 22 can be seen to have a set of therapeutic contours 24 on its dorsal surface 26. These replicate the contours 14 of the existing device 10 (see FIG. 1(b)). At the periphery of the replicated surface 26 is a zone 28 that has been pushed flat as a result of pressure applied as per the direction of the directional arrow. The line 30 establishes the datum from which the replicated angle β is measured. The angle is one of inversion, equivalent to that measured in FIG. 1(b).

Referring to FIG. 1(f), a reinforcing layer 32 is applied to the plantar surface 34 of the workpiece 22 to provide support and reinforcement against deformation of the contours 24. The layer 32 is stabilized against the plantar surface 34 by means of a phenolic resin adhesive 36 applied between the two opposed surfaces, to bond them together.

With reference to FIG. 1 (g), an optional filler material is shown adhered in an additional plantarly adhered layer 38 and additional adhesive layer 40. This filler layer retains a small, scalloped out void to its medial aspect.

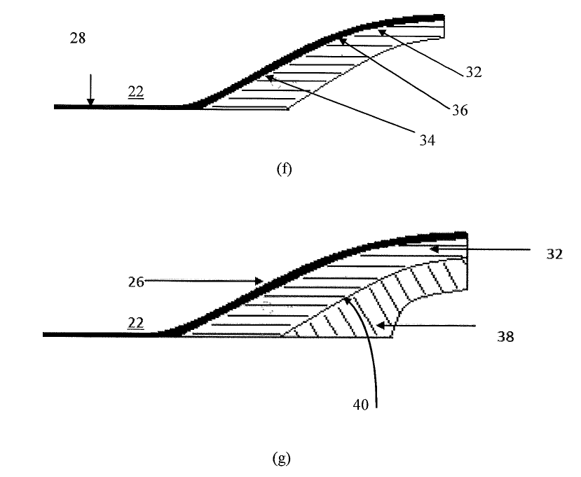

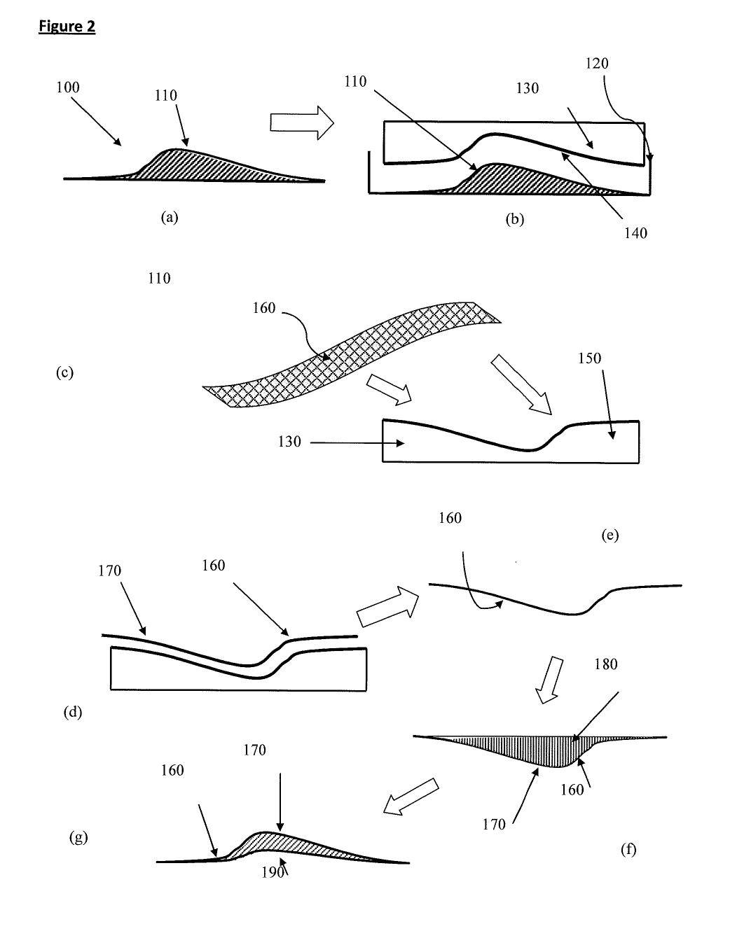

With reference to FIG. 2, there is shown in schematic form an alternative embodiment of the process of the invention. In FIG. 2(a), the therapeutically contoured profile 110 of an existing orthotic device 100, shown in medial view longitudinal-section, is desired to be replicated in a new device. In (b), the existing device 100 is shown placed in a moulding container 120 and covered with a mouldable substance 130, which in this example is a foam impression box. The moulding substance produces a cast by taking on an inverted form of the profile at its interfacial surface 140 that abuts the therapeutically contoured profile 110 to be replicated. The substance 130 is allowed to set to form a cast 150, which is then removed from the container 120 and inverted for the next step.

In part (c), a sheet of thermoplastics 160, which has been heated to soften to a malleable and re-settable state, is placed over the interfacial surface 140 of the cast 150, as suggested by the directional arrows. The sheet is allowed to cool to a set state in which it retains a profile corresponding to that of the interfacial surface 140, as shown in (d). In (e) the sheet 160 is peeled away from the cast 150, displaying the profile 170, corresponding to that of the original device 110.

In FIG. 2(f) a reinforcing layer with optional additional filler layer 180 is introduced into the concave hollow of the sheet 160 to provide support and strength for the sheet when subjected in use to pressure from the foot of the end user. In part (g), there is shown a void 190 machined into the filler 180 to adapt the composite device, now comprising the sheet, reinforcing layer and the filler, to fit complementally into a particular item of the user's footwear (not shown).

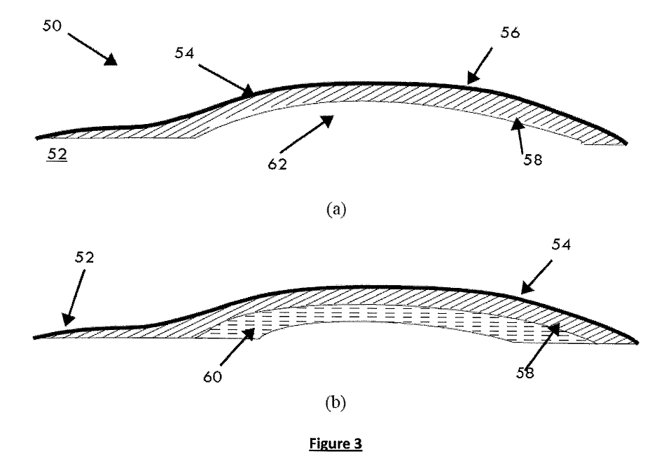

FIG. 3(a) shows an orthotic device 50 in longitudinal section as seen from a medial toward lateral view, thus showing the medial edge, this being the edge associated with the inner, arched border of the foot. The posterior or heel end is denoted by the number 52. The device has been manufactured according to the invention from a mould made of an existing orthotic, the mould having functioned as a template for forming the thin contoured shell 54 of the device having therapeutically contoured surface 56.

Also shown in FIG. 3 is a supporting layer 58. This may be made of ethylene vinyl acetate (EVA) and reinforces the functional layer, which permits its thickness to be minimised. Optionally, as shown in FIG. 3(b), a filler material 60 is added to the void 62 defined by the shaped plantar contour of the supporting layer 58 and its relation to the supporting surface. An example of this filler material 60 is the closed cell synthetic rubber product available under the trademark Poron® or the similar material known as PPT™.

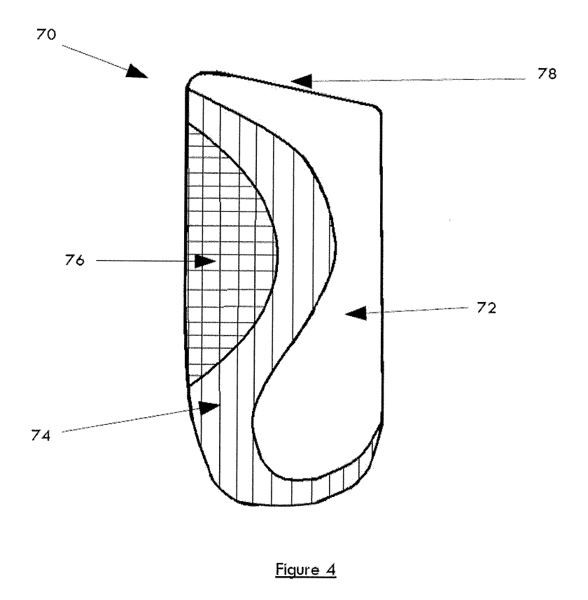

With reference to FIG. 4, an orthotic device 70 for use with the user's left foot and shown in plantar plan view, has a layer of EVA 74 that supports a 1.2 mm thick functional portion 72. Plantar to the EVA layer 74 is a void optionally filled with lower density material 76 such as PPT™, Poron® or a closed cell rubber. The anterior (forward) end is designated by the number 78.

In preferred embodiments of the invention, the orthotic device has a plantar surface that has been shaped to be complementary to the contours of the actual footwear item to which it is fitted. Therefore, during manufacture, access to the intended target footwear or a mould of its interior is desirable to enable it to be surveyed and consideration given to creating plantar contours that are complementary to the internal footwear dimensions and angles. Creating such a complementary interfacial shape between the device and the internal shoe void enhances mechanical stability of the device when worn, without adding bulk.

When the device is customized to match closely the internal dimensions of the footwear into which it has been designed to be inserted, and being so congruently matched and optionally adhered, the strength of the footwear materials to which it is aligned will desirably add to the strength of the device. This situation can be likened to adhering a contiguous sheath (this being the footwear) around the device, with the sides of the sheath possessing an independent ability to maintain their orthogonal opposition to the supporting surface, without the addition of another layer of material to further be accommodated within the footwear. The method thus includes shaping, for example by grinding, the plantar surface to be congruent with the interior surface of the target footwear. Alternatively, or in addition, it may be deliberately shaped to be incongruous in an intended way so as to produce a predetermined therapeutic effect, for example, creating a medial arch void to increase device flexibility.

From the embodiments described above, it will be appreciated that this invention provides for the manufacture of an orthotic device of a single layer or of a composite, including multi-layered, construction, the choice being influenced by factors such as the required function of the orthotic device, the particular shoe or other item of target footwear with which it is to be used, the weight of the user, the level of activity envisaged for the user and the nature and availability of materials for the layers.

The following are some advantages of the invention:
The manufacturing process of the invention expands the category of persons who are empowered to manufacture orthotic devices. It allows technicians (rather than allied health professionals only) to use documented processes incorporating quality control standards to construct customised orthotic devices. This presents potential cost savings, which can be passed on to the user, thereby increasing the incidence of usage of orthotic devices and the frequency with which they are upgraded and replaced.
The reduced bulk of the orthotic product that can result from the practicing of the invention means a significant increase in the proportion of footwear items that will be able to accept a customised insert. Prior to this invention there was a limited range of shoes available to the wearers of the orthotics of the prior art, even when these were customised directly according to the actual foot of the user.
The invention provides a customised orthotic device without requiring manufacturer access to the foot of the individual patient requiring the service. Normally, lack of physical proximity of the patient to the service provider would result in a lack of individualisation.
The process of the invention lends itself to the use of highly mouldable and lightweight materials not found in prior art orthotic devices, the resultant device being therefore smaller and lighter and increasing the comfort of the user.
Fitting orthotics of the prior art to aged persons and in particular to those with a lack of natural subdermal foot padding, or those with deforming diseases such as rheumatoid arthritis, osteoarthritis and diabetes mellitus—has previously been problematic, as the hard nature of the traditional thermoplastic devices may cause discomfort in such users. The invention allows the firmness/softness and flexibility/rigidity of the device to be customised to any level that is prescribed for or desired by the user.
In cases where the malleable material possessed of contour memory is not sufficiently strong to withstand use as an in-shoe orthotic on its own, it is useful in forming a template and then being incorporated into the complete orthotic device by using layering materials to fortify the finer template material.
The bulk of all non-essential areas of the orthotic, namely the peripheral zone, is pared down and reduced to thicknesses less than 2 mm, improving footwear choices.
The invention enables manufacture of an orthotic device that may be fitted or adapted to fit with an expanded range of the user's footwear, vastly expanded from the footwear choices available to most orthotic wearers. This improves the level of client contentment and allows personal fashion preference to be entertained in the choice of the client's footwear.
Expanding on the previous advantage, it will be appreciated that orthotic imposed footwear restrictions have previously either severely restricted shoe choices or diminished the user's ability to wear the therapeutic orthotic device. As orthotic devices are generally prescribed to either reduce current pain or protect against future damage to body tissues (for example joint wear and tear), the inability to wear them when required imposes a cost on the individual and possibly on the health system of the user's country, where such systems exist.
The diagnostic responsibilities of health care professionals and, in particular, podiatrists, remain essential to the foot care process, as the device of the invention is suitable for use when an individual has an orthotic device that performs its function properly but fails to fit the patient's preferred footwear.
In making an orthotic device according to the invention, the maker need only have knowledge of the inventive process: No knowledge associated with foot function, diagnosis, disorders or treatment is required. This has the potential to make this service broadly available and at a lower cost than a consultation with a health service provider, as a competent technician versed in the inventive process will suffice.
The inexpensive nature of the inventive device relative to a customised orthotic device of the prior art will allow better customisation of the device to the intended activity of the user—and thus improved specificity—as the wearer may acquire multiple orthotic devices each designed for one of a range of applications, rather than a single device that is required to perform under a multitude of circumstances.
The device of the inventive process can be designed to be an enduring product with a lifespan similar to that of a common shoe. However, the lifespan will be dependent upon the materials used to make the device and the choice of material in turn is dependent upon the client's shoe selection, medical specifications personal desires and preferences. The continuum of possibilities would range from a very soft material, which provides lesser support and greater comfort but a relatively short lifespan, to a highly durable, maximally controlling material with a relatively long lifespan.

The process of the invention described herein offers a variety of alternative materials for the construction of an orthotic device. Whilst some alternatives may infer a permanent fixation of the device to a footwear item, such as a shoe, a removable product is also contemplated and is achieved by using non-adhesive components for forming the surfaces that make contact with the interior of the footwear.

These embodiments merely illustrate particular examples of the invention and the method of using it in making an orthotic insertion for a footwear item. With the insight gained from this disclosure, the person skilled in the art is well placed to discern further embodiments by means of which to put the claimed invention into practice. It should thus be appreciated that changes may be made to various features in the above described embodiments without departing from the spirit and scope of the invention, and that the invention is not limited to the specific embodiments described herein.

1-39. (canceled)

40. A method of manufacturing an orthotic device for use with a desired item of footwear, the method comprising the steps of providing the desired footwear item, providing an existing orthotic device that has an existing, therapeutically-shaped contoured surface, using said existing surface as a template in forming a body that has a contoured surface that replicates said existing surface substantially entirely and adapting the body to be customized for fitting said desired item of footwear and for user activity for which the footwear is intended.

41. The method of claim 40 wherein the body is formed by applying a shape-capturing material in sheet form to said existing surface and allowing it to set to a self-supporting state, so that a surface of the sheet, which is not in contact with the existing surface, defines a dorsal surface that replicates said existing surface.

42. The method of claim 41 comprising adapting the sheet to define an orthotic device customised to fit a foot-receiving space of the desired item of footwear.

43. The method of claim 41 wherein the shape-capturing material is a thermoplastics material.

44. The method of claim 41 including separating the sheet, when in a self-supporting state, from the surface to which it has been applied, the separated surface of the sheet defining a plantar surface.

45. The method of claim 44 including affixing a second, thin sheet of malleable shape-capturing material to the plantar surface, allowing said second sheet to set to a self-supporting state so that a surface of said second sheet, as a result of being in contact with the plantar surface, defines a dorsal surface that replicates said existing surface and said sheets thereby in combination define a second orthotic device having opposing dorsal and plantar surfaces, and adapting said second device to fit a specified item of footwear of the user.

46. The method of claim 45 wherein the second, thin sheet material is a thermoplastics material.

47. The method of claim 44 wherein the step of adapting the sheet includes identifying an area of said existing orthotic device that is not therapeutically essential to the functioning of the device and identifying a corresponding area of the sheet, so as to define a peripheral zone thereof.

48. The method of claim 47 including diminishing the peripheral zone to a minimum practical thickness.

49. The method of claim 47 including reducing the surface area of the peripheral zone.

50. The method of claim 47 further comprising providing the peripheral zone with a profile that tapers from being relatively thick where it merges with the therapeutically-shaped surface, to being relatively thin at an opposed exterior edge.

51. The method of claim 47 including affixing supporting means to the plantar surface of the sheet.

52. The method of claim 51 wherein the supporting means is selected to resist flexion of the sheet under force exerted by the weight of the user.

53. The method of claim 52 wherein the supporting means comprises a reinforcing layer.

54. The method of claim 53 including filling with a filler, at least partially, a void that would otherwise exist between the plantar surface of the device and a surface of the desired footwear when in use.

55. The method of claim 54 wherein the filler comprises a closed cell polymer.

56. A custom-made orthotic device comprising a sheet of shape-retaining material having a dorsal surface formed to replicate a therapeutically-functional shape defined by a dorsal surface of an existing orthotic device which provided a therapeutic benefit to an intended user of said custom-made device, said custom-made device being adapted to fit a foot-receiving space in a specific, desired item of footwear and to derive structural strength from the structure of the desired footwear.

57. The device of claim 56 wherein the sheet thickness is in the range from 0.8 to 2.5 mm.

58. The device of claim 56 wherein the sheet is substantially uniform in thickness.

59. The device of claim 56 wherein the sheet has a peripheral zone corresponding to a therapeutically non-essential area of the existing orthotic device.

60. The device of claim 59 wherein the peripheral zone has a profile tapering from being relatively thick where it merges with the therapeutically shaped surface to being relatively thin at an opposed exterior edge.

61. The device of claim 60 wherein the thickness of the peripheral zone is less than about 2 mm.

62. The device of claim 61 wherein the dorsal surface of the device has been replicated by having been placed in direct contact with the surface to be replicated.

63. The device of claim 56 having width not exceeding the width of the desired item of footwear.

64. The device of claim 56 manufactured to have user-preferred properties selected from hardness, softness, flexibility and rigidity.

65. The device of claim 57 comprising adhesion means for inhibiting its movement within the foot-receiving space in use.

66. The device of claim 56 further having supporting means located in abutment to the sheet.

67. The device of claim 66 wherein the supporting means comprises a reinforcing layer for the sheet.

68. A method of manufacturing a new orthotic device, the method comprising the steps of providing an item of footwear with which an intended user desires to use the new device, determining design parameters for the new device to enable use thereof with the desired footwear, providing an existing orthotic device used by said user and having a therapeutically-shaped dorsal surface, providing a sheet of malleable material, replicating the therapeutically-shaped surface of the existing orthotic device permanently on a surface of said sheet and adapting the shaped sheet to fit specifically with the desired item of footwear according to the user's preference, to define the new device.

69. The method of claim 68 wherein the design parameters are selected from shape, configuration, structure, materials, dimensions and combinations thereof.

70. The method of claim 68, wherein the step of determining the parameters comprises collecting data relating to one or more of the size, shape and configuration of the desired footwear item, size and weight of the user and the intended activities for which the device will be used.

71. The method of any one of claims 68, wherein the step of adapting the sheet includes one or more of trimming its size, adding a supporting portion, reducing its bulk and adding filler material between the composite reinforced device and a surface of the desired footwear item.

72. A custom-made orthotic device for insertion into a user's footwear, the device comprising a body formed from a sheet of self-supporting material having a therapeutically contoured dorsal surface and a lower portion adapted to fit the foot-receiving space of a particular item of footwear of a user and to the user's intended activity when wearing said footwear, wherein the dorsal surface replicates the therapeutic shape of a prior orthotic device of the user as a result of direct contact having been established during shaping of said surface with a therapeutically-shaped dorsal surface of said prior orthotic device.

73. A custom-made orthotic device for insertion into a user's footwear, the device comprising a body formed from a sheet of self-supporting material having a therapeutically contoured dorsal surface and a lower portion adapted to fit the foot-receiving space of a particular item of footwear of a user and to the user's intended activity when wearing said footwear, wherein the dorsal surface replicates the therapeutic shape of a prior orthotic device of the user as a result of direct contact having been established during shaping of said surface with a surface derived by way of a cast from a therapeutically-shaped dorsal surface of said prior orthotic device.

74. The device of claim 73 further comprising a comfort-providing and cosmetic layer applied to the dorsal surface.
